(19) United States
(12) Patent Application Publication
(10) Pub. No.: US 2015/0247196 A1
(43) Pub. Date: Sep. 3, 2015

(54) IN VITRO GENETIC DIAGNOSTIC OF INHERITED NEUROMUSCULAR DISORDERS

(71) Applicants: INSERM (INSTITUT NATIONAL DE LA SANTÉ ET DE LA RECHERCHE MÉDICALE), Paris (FR); UNIVERSITÉ D'AUX-MARSEILLE, Marseille (FR); CENTRE NATIONAL DE LA RECHERCHE SCIENTIFIQUE - CNRS, Paris Cedex 16 (FR); UNIVERSITE MONTPELLIER I, Montpellier (FR)

(72) Inventors: Pascal Soularue, Evry (FR); David Atlan, Blonay (CH); Valerie Allamand, Paris (FR); Marc Bartoli, Marseille (FR); Christophe Beroud, Marseille (FR); Gisele Bonne, Paris (FR); Patrice Bourgeois, Marseille (FR); Sebahattin Cirak, Washington, DC (US); Mireille Cossee, Montpellier (FR); Rafael De Cid, Badalona (ES); Martin Krahn, Marseille (FR); Nicolas Levy, Marseille (FR); Francesco Muntoni, London (GB); Isabelle Richard, Corbeil (FR)

(21) Appl. No.: 14/426,553

(22) PCT Filed: Sep. 6, 2013
(86) PCT No.: PCT/EP2013/068510

§ 371 Date: Mar. 6, 2015

(30) Foreign Application Priority Data
Sep. 7, 2012 (EP) 12306081.6

(51) Int. Cl. C12Q 1/68

(52) U.S. Cl. CPC C12Q 1/6883; C12Q 2600/156; C12Q 2600/16

(57) ABSTRACT

The present invention provides a method for determining all the molecular causes of Inherited Neuromuscular Disorders comprising determining a number of copy number variation(s), and/or determining a number of point mutation(s) on a physiological sample comprising a genome of a subject.

### FIELD OF THE INVENTION

The present invention relates to an in vitro method, the preparation and the application thereof, for determining Inherited Neuromuscular Disorders, more rapidly with more accuracy and less cost.

### BACKGROUND OF THE INVENTION

Inherited Neuromuscular Disorders (hereafter NMDs) form a large and very heterogeneous group of genetic diseases that cause progressive degeneration of the muscles and/or motor nerves that control movements. Most NMD types result in chronic long term disability posing a significant burden to the patients, their families and public health care. Life is usually shortened by multiple and cumulative defects that occur during disease progression. Premature death may result from cardiac and respiratory muscle involvement. Namely pathologies such as Muscular Dystrophies (hereafter MD), limb girdle muscular dystrophies (hereafter LGMD), congenital muscular dystrophies (hereafter CMD), and other neuromuscular diseases such as congenital myopathies (hereafter CM) and Duchenne/Becker Muscular Dystrophies (DMD/BMD) represent a large proportion of NMDs.

These pathologies are present in all populations, affecting children as well as adults. The overall prevalence of NMDs is very difficult to evaluate, but one can estimate that, given the incidence of every different type, around 1 out of 1000 people may have a disabling inherited neuromuscular disorder.

The precise determination of NMDs requires a conjunction of extensive clinical examination and targeted complementary tests: biological analyses, electromyography, imaging, and histological analysis of biopsies. Numerous genes containing disease causing mutations responsible for NMDs are known; thus molecular genetic analyses are performed both to confirm the clinical diagnosis and to precise the genotype of each patient. However, one cannot avoid the difficulties in making a molecular determination of these diseases, due to frequent overlaps of clinical phenotypes, the large number of known genes, large genes that lack “hot spots” of mutations, etc. . . . . As a consequence, according to reliable estimate, 30 to 40% of patients remain devoid of genetic confirmation of their disease type, although disease causing mutation lies in an already known gene.

Most of the molecular approaches currently used for genetic analysis correspond to gene by gene explorations, starting by the most pertinent one.

As shown in FIG. 1, in the current state of the art diagnostic process, in most cases, two kinds of samples are necessary: muscle (and/or skin) biopsy sample and blood sample.

NMDs analysis is carried out on DNA extracted from blood samples according to the following steps:

(1) a molecular analysis of the most pertinent gene 1 is implemented, in order to identify abnormality on said gene 1; and
(2) if no abnormality is detected, after a clinical consultation, a molecular analysis is implemented on the second most pertinent gene 2, in order to search a possible abnormality on said gene 2.

The above steps are repeated for pertinent genes in decreasing order of pertinence, until a mutation is detected, giving a genetic confirmation of disease, before turning to therapy phase.

The total time necessary for finding the mutation can be up to one year, or even more for some cases.

Thus with the presently available technologies, a differential molecular genotyping is required, which is highly complex and time consuming (some weeks to one year).

Many patients thus remain devoid of genetic confirmation of their disease. To date, this proportion amounts to 30 to 40% of patients suffering of NMDs. Importantly, new cutting edge therapies, such as exon skipping, cannot be contemplated if no precise genetic analysis is available.

Piluso et al. (1) disclose a comparative genomic hybridization microarray for copy number variations in 245 genes and 180 candidate genes implicated in NMDs.

Even though the method of Piluso et al. allows detecting copy number variations, said method does not allow detecting all the molecular causes of Inherited Neuromuscular Disorders.

It would therefore be desirable to provide a method for determining all the molecular causes of Inherited Neuromuscular Disorders more rapidly, with more accuracy and less cost.

### SUMMARY OF THE INVENTION

The applicant has now found that such an aim is achieved by a in vitro method of identifying molecular causes for at least one of MD, LGMD, CMD and other neuromuscular diseases.

As used herein, “molecular causes” indicates mutations due to Copy Number Variations (hereafter CNVs) and point mutations.

As used herein, “other neuromuscular diseases” indicate all the neuromuscular diseases mentioned in the gene table of Kaplan (2), other than MD, LGMD and CMD.

More precisely, the present invention relates to a method of identifying in vitro molecular causes of Inherited Neuromuscular Disorders, comprising the following steps:

(i) providing a physiological sample comprising a genome of a subject, and
(ii) implementing on said sample at least one of Process A and Process B, wherein:
Process A is determining a number of copy number variation(s), with respect to a sample of a normal subject, on at least 25 genes selected from the 31 genes of Group 1 in Table 1 and
Process B is determining a number of point mutation(s) with respect to a sample of a normal subject on at least 25 genes selected from the 31 genes of Group 1 in Table 1.

In order to detect at least one of MD, LGMD, CMD and other neuromuscular diseases, it is mandatory to detect all different mutations in the targeted genes.

Two types of gene mutations are involved in said NMDs:
1. CNV, and
2. Point mutation.

As used herein, “Copy Number Variation (CNV)”, is an alteration of the DNA of a genome, resulting in an abnormal number of copies of one or more sections of the DNA. CNV can be a gain or a loss of specific DNA sequence(s) in DNA, such as deletions, duplications or amplifications of sequence(s).

As used herein, “point mutation” is replacement of single base nucleotide with another nucleotide of the genetic material.

As used herein, “a normal subject” indicates a subject who is devoid of any neuromuscular disease.

Process A allows determining a number of CNV(s) on at least one of the targeted genes of Table 1, and thus determining a number of at least one of MD, LGMD, CMD and other neuromuscular diseases arising from CNV(s).

In one embodiment of a method of identifying in vitro molecular causes of Inherited Neuromuscular Disorders of the present invention, step (ii) comprises only Process A.

Process A however does not allow detecting (a) point mutation(s) on said genes.

Process B allows detecting (a) point mutation(s) on said genes of Table 1, and thus determining a number of at least one of MD, LGMD, CMD and other neuromuscular diseases arising from (a) point mutation(s).

In another embodiment of a method of identifying in vitro molecular causes of Inherited Neuromuscular Disorders of the present invention, step (ii) comprises only Process B.

Therefore, it is interesting to implement Process B in order to detect (a) point mutation(s) on said genes, thus enabling to determine a number of at least one of MD, LGMD, CMD and other neuromuscular diseases with a higher rate of determination with respect to a well known technique of the prior art and any conventional technique.

Particularly, since Process A and Process B are complementary, their combined use allows determining all possible molecular causes on said genes with a high determination rate.

In a preferred embodiment of a method of identifying in vitro molecular causes of Inherited Neuromuscular Disorders of the present invention, step (ii) comprises Process A and Process B.

Table 1 hereafter presents 62 genes which have been identified, after extensive research on NMDs, to be involved in at least one of MD, LGMD, CMD and other neuromuscular diseases.

The definition of each gene can be found on http://genome.ucsc.edu.

TABLE 1

| | | | |
|---|---|---|---|
| ACTA1 | BAG3 | CFL2 | CHKB |
| ANO5 | DAG1 | DNAJB6 | CNTN1 |
| BIN1 | LAMP2 | FKTN | ICSU |
| CAPN3 | LDB3 | GDF8 | ITGA7 |
| CAV3 | MTM1 | KLH9 | MYBPC3 |
| COL6A1 | MYH7 | MYH2 | PLEC1 |
| COL6A2 | NEB | POMGNT1 | TCAP |
| COL6A3 | PABPN1 | SGCB | Group 4 |
| DES | PTRF | SGCD | |
| DMD | SYNE1 | Group 3 | |
| DNM2 | SYNE2 | | |
| DOK7 | TNNT1 | | |
| DYSF | TPM2 | | |
| EMD | TPM3 | | |
| FHL1 | VCP | | |
| FKRP | Group 2 | | |
| FLNC | | | |
| GNE | | | |
| KRYAB | | | |
| LAMA2 | | | |
| LARGE | | | |
| LMNA | | | |
| MYOT | | | |
| POMT1 | | | |
| POMT2 | | | |
| RYR1 | | | |
| SEPN1 | | | |
| SGCG | | | |
| SGCA | | | |
| TRIM32 | | | |
| TTN | | | |
| Group 1 | | | |

In Table 1, 31 genes are classified as Group 1 corresponding to the highest level of implication, 15 genes are classified as Group 2 corresponding to a higher level of implication, 9 genes are classified as Group 3 corresponding to a high level of implication, and 7 genes are classified as Group 4 corresponding to a certain level of implication, in at least one of MD, LGMD, CMD and other neuromuscular diseases

Table 2 shows the implication of the genes of Table 1 in each disease.

TABLE 2
61 genes of Table 1 classified by implications in each of NMDs

| | | | | | | | | Other | | |
|---|---|---|---|---|---|---|---|---|---|---|
| | | | | | | | | neuro | | |
| | | | | | | | | muscular | | |
| MD | | LGMD | | | CMD | | | diseases | | |
| A | B | A | B | C | A | B | C | A | B | C |
| DMD | FHL1 | CAV3 | DNAJB6 | TCAP | LAMA2 | POMGTN1 | TCAP | ACTA1 | CFL2 | ICSU |
| EMD | SYNE1 | LMNA | SGCB | PLEC1 | POMT1 | FKTN | ITGA7 | SEPN1 | MTM1 | |
| LMNA | SYNE2 | MYOT | SGCD | | POMT2 | FHL1 | CHKB | TPM2 | MYH2 | |
| GNE | PTRF | CAPN3 | POMT1 | | FKRP | DMN2 | MYBPC3 | TPM3 | KLH9 | |
| DOK7 | | DYSF | POMT2 | | LARGE | MYOT | CNTN1 | NEB | GDF8 | |
| | | ANO5 | POMGNT1 | | COL6A1 | KLH9 | TRIM32 | TNNT1 | FHL1 | |
| | | SGCG | FKTN | | COL6A2 | | | RYR1 | | |
| | | FKRP | | | COL6A3 | | | MYH7 | | |
| | | TTN | | | SEPEN1 | | | DNM2 | | |
| | | TRIM32 | | | TTN | | | BIN1 | | |
| | | DAG1 | | | FLNC | | | MTM1 | | |
| | | SGCA | | | LMNA | | | VCP | | |
| | | | | | | | | KRYAB | | |
| | | | | | | | | DES | | |
| | | | | | | | | FLNC | | |
| | | | | | | | | MYOT | | |
| | | | | | | | | LDB3 | | |
| | | | | | | | | BAG3 | | |
| | | | | | | | | LAMP2 | | |
| | | | | | | | | PABPN1 | | |

In Table 2, the genes implicated in each disease are classified according to their level of implication: the genes classified as A correspond to the highest level of implication, the genes classified as B correspond to a higher level of implication, and the genes classified as C correspond to a high level of implication.

### DESCRIPTION OF THE PREFERRED EMBODIMENTS

As shown in FIG. 2, providing a blood sample is sufficient to implement the method of the present invention.

The present invention relates to a method of identifying in vitro molecular causes of Inherited Neuromuscular Disorders, comprising the following steps:

(i) providing a physiological sample comprising a genome of a subject, and
(ii) implementing on said sample at least one of Process A and Process B, wherein:
Process A is determining a number of copy number variation(s), with respect to a sample of a normal subject, on at least 25 genes selected from the 31 genes of Group 1 in Table 1, and
Process B is determining a number of point mutation(s), with respect to a sample of a normal subject, on at least 25 genes selected from the 31 genes of Group 1 in Table 1.

In one embodiment of a method of identifying in vitro molecular causes of Inherited Neuromuscular Disorders of the present invention, step (ii) comprises only Process A.

In another embodiment of a method of identifying in vitro molecular causes of Inherited Neuromuscular Disorders of the present invention, step (ii) comprises only Process B.

In a preferred embodiment of a method of identifying in vitro molecular causes of Inherited Neuromuscular Disorders of the present invention, step (ii) comprises both Processes A and B.

As used herein, “targeted genes” signifies the genes on which Process A or Process B is, or Process A and Process B are carried out.

Process A allows determining a number of CNV(s) on at least 25 genes selected from the 31 genes of Group 1 in Table 1.

Process B allows determining a number of point mutation(s) on at least 25 genes selected from the 31 genes of Group 1 in Table 1.

When the number of targeted genes increases, the determination rate of at least one of MD, LGMD, CMD and other neuromuscular diseases is increased.

When step (ii) comprises both Process A and Process B, said at least 25 genes of Group 1 for Process A and Process B can be selected dependently of independently from said at least 25 genes of Group 1 for Process B. In other terms, the genes used in Process A and Process B may be the same or may be partly or totally different from each other.

In one preferred embodiment, Process A or Process B is, or Process A and Process B are carried out on all the 31 genes of Group 1 in Table 1.

In a more preferred embodiment, Process A or Process B is, or Process A and Process B are carried out on all the 31 genes of Group 1, and on at least 10, preferably all the 15 genes of Group 2 in Table 1.

When step (ii) comprises both Process A and Process B, said at least 10 genes of Group 2 for Process A and Process B can be selected dependently or independently from said at least 10 genes of Group 2 for Process B. f

In an even more preferred embodiment, Process A or Process B is, or Process A and Process B are carried out on all the 31 genes of Group 1, on all the 15 genes of Group 2, and on at least 5, preferably all the 9 genes of Group 3 in Table 1.

When step (ii) comprises both Process A and Process B, said at least 5 genes of Group 3 for Process A and Process B can be selected dependently or independently from said at least 5 genes of Group 3 for Process B.

In a most preferred embodiment, Process A or Process B is, or Process A and Process B are carried out on all the 31 genes of Group 1, on all the 15 genes of Group 2, on all the 9 genes of Group 3, and on at least 4, preferably all the 7 genes of Group 4 in Table 1.

When step (ii) comprises both Process A and Process B, said at least 4 genes of Group 4 for Process A and Process B can be selected dependently or independently from said at least 4 genes of Group 4 for Process B.

In another most preferred embodiment providing an excellent rate of detection, Process A or Process B is, or Process A and Process B are carried out on all the genes of the Table 3 hereunder:

TABLE 3

| | | | |
|---|---|---|---|
| ACTA1 | FKRP | BAG3 | DNAJB6 |
| ANO5 | FLNC | DAG1 | FKTN |
| BIN1 | GNE | LAMP2 | MYH2 |
| CAPN | LAMA2 | LDB3 | SGCB |
| CAV3 | LARGE | MTM1 | SGCD |
| COL6A1 | LMNA | NEB | CHKB |
| COL6A2 | MYOT | PABPN1 | CNTN1 |
| COL6A3 | POMT1 | PTRF | ITGA7 |
| DES | POMT2 | TNNT1 | PLEC1 |
| DMD | RYR1 | TPM2 | TCAP |
| DNM2 | SGCG | TPM3 | |
| DYSF | SGCA | VCP | |
| FHL1 | TTN | CFL2 | |

If one or more CNVs or one or more punctual mutations are(is) detected, the physiological sample comprising a genome of a subject is classified as positive and a precise type of at least one of MD, LGMD, CMD and other neuromuscular diseases is allotted to said sample.

Process A consists in
providing a physiological sample comprising a genome of a subject, and
determining a number of CNV(s) on targeted genes of the genome of the sample.

From the CNV(s) detected on said targeted genes, a person skilled in the art can determine a number of at least one of MD, LGMD, CMD and other neuromuscular diseases arising from CNV(s) on said genes.

For example, if a CNV is detected on gene POMT1, which is involved in both LGMD and CMD (c.f. Table 2), said sample has LGMD or CMD arising from a CNV.

Here, Process A may be implemented by any well known technique of the prior art and any conventional technique allowing determining a number of CNV(s).

In one embodiment, Process A is carried out with a Device A comprising a set of probes for said targeted genes.

In one particular embodiment, said Device A is a Comparative Genomic Hybridization (CGH) array.

Therefore, for example, by using CGH technique, Process A consists in
providing a physiological sample comprising a genome of a subject,
providing a CGH array provided with a set of probes for at least 25 genes selected from the 31 genes of Group 1 in Table 1,
analysing said sample using said CGH array, and
determining a number of CNV(s) in the DNA of the sample.

A suitable physiological sample may be for example a biopsy sample, whole blood, a lymphocyte culture, preferably, whole blood or a lymphocyte culture, particularly preferably a lymphocyte culture.

One usual blood sampling provides an amount of sample sufficient for implementing the method of the present invention.

As used herein, “Comparative Genomic Hybridization (CGH)” is a type of nucleic acid hybridization assay which detects and identifies the location of CNV. The technique is described in detail, for example in references (3), (4) and (5).

CGH is a co-hybridization assay of differentially labelled test DNA (for example green fluorescent dye) and reference DNA (for example red fluorescent dye) that includes the following major steps:

(1) immobilization of nucleic acids to a support to provide an immobilized probe;

(2) pre-hybridization treatment to increase accessibility of the probe and to reduce nonspecific binding;

(3) hybridization of a mixture of target nucleic acids to the probe;

(4) post-hybridization washing to remove nucleic acid fragments not hybridized to the probe; and
(5) determination of the target nucleic acids hybridized to the probes using a determination device.

The 62 genes of Table 1 are involved in at least one of MD, LGMD, CMD and other neuromuscular diseases.

Device A comprises a set of probes for at least 25 genes selected from of the 31 genes of Group 1 on Table 1.

When the number of targeted genes increases, the determination rate of at least one of MD, LGMD, CMD and other neuromuscular diseases is increased.

Therefore in one preferred embodiment, said Device A comprises a set of probes for the 31 genes of Group 1 in Table 1, and for at least 10, preferably all the 15 genes of Group 2 in Table 1.

In an even more preferred embodiment, said Device A comprises a set of probes for all the 31 genes of Group 1, for all the 15 genes of Group 2, and for at least 5, preferably all the 9 genes of Group 3 in Table 1.

In a most preferred embodiment, said Device A comprises a set of probes for all the 31 genes of Group 1, for all the 15 genes of Group 2, for all the 9 genes of Group 3, and for at least 4, preferably all the 7 genes of Group 4 in Table 1.

CNV(s) in a tested DNA of the sample can be determined in the following manner, using two colour labels:

In case of a deletion in the tested DNA, less tested DNA will bind to the corresponding spots and a first colour label of the reference DNA will prevail. Gains in the tested genome can be identified by a dominance of second colour label of the tested DNA. Spots representing sequences with the same copy number in the tested genome relative to the reference genome are revealed by a third colour.

Thus, the ratio fluorescence intensity of the tested DNA/fluorescence intensity the reference DNA is then calculated, in order to measure the copy number changes for a particular location in the genome.

As used herein, “a set of probes” means a set of fragments of nucleotides having sequences capable of hybridizing with the sequence of the genes to be analysed.

A person skilled in the art can prepare a suitable set of probes for said Device A, by any well known technique of the state of the art, such as the method described by reference (6).

In one embodiment, said set of probes for said Device A comprises:
probes evenly spaced by about 50 bp distance between two consecutive probes, which hybridize said gene plus a region of about 2000 bp at the 5′ and 3′ terminal exons, and
backbone probes not evenly spaced by about 1000 bp distance between two consecutive probes, which represent on average from 20 to 30% of the total probes on said device.

As used herein, “evenly spaced” indicates “spaced by a same interval”.

As used herein, “backbone probes” are probes which hybridize to locations on the genome going beyond the genes of interest, such as intronic and potentially intergenic regions. They are used to generate a calibration signal against which the test and reference signals from the specific gene probes are measured.

As used herein, “covered” indicates “capable of hybridizing with the sequence of the genes to be analysed”.

In one particularly preferable embodiment, said set of probes are manufactured according to the following rules:

probes cover gene+/−2 kb up and downstream,

average probe density is 1/50,

probes are alternated on (+) and (−) strands,

with a tiling of
10 pb tiling in exonic regions and intron-exon boundaries (150 bp upstream and downstream of the exon)
30 pb tiling in 3′ and 5′ UTR
one probe for 100 bp in introns

backbone probes each 6 kb,

total number of probes: 137207,
gene probes (exon, intron, 5′ and 3′-UTR): 69570

backbone probes: 67637 probes (one each 6 kb on average).

As used herein, “average probe density” indicates the inverse of the mean distances between the start positions of consecutive probe sequences on the indicated region of the genome:

$$\frac{n - 1}{{\sum }_{i = 1}^{i = n - 1} [ ( [ {distance ( Probe ]}_{i} , {Probe}_{i + 1} ) ] )}$$

where n is the number of probes in the considered region of the genome.

As used herein, “tiling” indicates the mean distance between consecutive probes. 1/Tiling represents the average probe density.

This probe design allows increasing the robustness of the determination of the present invention.

Process B consists in
providing a physiological sample comprising a genome of a subject, and
determining a number of point gene mutation(s) on targeted genes of the genomes of the sample.

From said point mutation(s) detected on said targeted genes, a person skilled in the art can determine a number of at least one of MD, LGMD, CMD and other neuromuscular diseases arising from (a) point mutation(s) on said genes.

For example, if a point mutation is detected on gene POMT1, which is involved in both LGMD and CMD (c.f. Table 2), said sample has LGMD or CMD arising from a point mutation.

Here, Process B may be implemented by any well known technique of the prior art and any conventional technique allowing determining a number of point mutation(s).

In one embodiment, Process B is carried out with a Device B comprising a set of probes for said genes.

In one embodiment, Process B is carried out by a technique selected from the group consisting of Sequence capture, “on-chip capture” and “in-solution capture (Sure Select)”.

In one particular embodiment, Device B is a Sequence capture array.

Therefore, for example, Process B consists in
providing a physiological sample comprising a genome of a subject,
providing a sequence capture array provided with a set of probes for at least 25 genes selected from the 31 genes of Group 1 on Table 1,
analysing said sample using said sequence capture array, and
determining a number of punctual mutation(s) in the DNA of the sample.

For Process B, the same physiological sample as that prepared for Process A may be used.

The 62 genes of Table 1 are involved in at least one of MD, LGMD, CMD and other neuromuscular diseases.

Said Device B comprises a set of probes for at least 25 genes selected from the 31 genes of Group 1 on Table 1.

When the number of targeted genes increases, the determination rate of a number of at least one of MD, LGMD, CMD and other neuromuscular diseases is increased.

In one preferred embodiment, said Device B comprises a set of probes for the 31 genes of Group 1 in Table 1, and for at least 10, preferably all the 15 genes of Group 2 in Table 1.

In one more preferred embodiment, said Device B comprises a set of probes for all the 31 genes of Group 1, for all the 15 genes of Group 2, and for at least 5, preferably all the 9 genes of Group 3 in Table 1.

In one more preferred embodiment, said Device B comprises a set of probes for all the 31 genes of Group 1, for all the 15 genes of Group 2, for all the 9 genes of Group 3, and for at least 4, preferably all the 7 genes of Group 4 in Table 1.

“DNA sequence capture” consists in isolating and sequencing a genomic region of interest (targeted region), to the exclusion of the remainder of the genome, and then sequencing the captured DNA fragments.

“Sequencing” means determining the sequence of target DNA fragments.

DNA sequence capture includes the following major steps:

(1) preparation of a sequencing library of test DNA;

(2) hybridization of said sequencing library to a support with immobilized probes thereon;

(3) post-hybridization washing to remove nucleic acid fragments not hybridized to the probe;

(4) target DNA fragment elution;

(5) PCR amplification of target DNA fragment; and

(6) sequencing of target DNA fragment.

In the present invention, the term “target regions” indicates regions of a gene which are especially involved in at least one of MD, LGMD, CMD and other neuromuscular diseases.

A person skilled in the art can manufacture a suitable set of probes for said Device B by any well known technique of the prior art and any conventional technique, such as the method described by reference (6).

In one embodiment, said set of probes for said Device B comprises:

probes of 70 to 120 bp, which hybridize all the exons of said genes with at least 2× tiling frequency.

As used herein, “tiling frequency” indicates the density of tiling. For example, 2× tiling frequency means that each base is covered by two different probes.

In one embodiment, said set of probes for said Device B for Process B is prepared according to the following rules:

exonic regions apart from 3′UTR are covered,

exonic regions and intron-exon boundaries (200 bp upstream and down stream of the exon) are covered,

1 kb upstream and downstream of each gene (5′ and 3′ UTR) are covered, and

probes are alternated on (+) and (−) strands.

This probe design allows increasing the robustness of the determination of the present invention.

Device B of the present invention preferably comprises an array or solid particles suspended in liquid such as magnetic particles.

In one embodiment, High Throughput Sequencing (hereafter HTS) is used in Process B, for allowing further lowering the cost of analysis.

Device A may be manufactures as follows:

The sequences of targeted genes known to be responsible for at least one of MD, LGMD, CMD and other neuromuscular diseases were obtained from the web site of the UCSC (http://genome.ucsc.edu), and are shown in Table 1.

The genes on which analysis is implemented are selected from Table 1, as explained above.

A set of probes for Device A can be designed by a well known technique of the state of the art, as explained above.

For example, CGH arrays which may be used in Process A may be manufactured by any manufacturer specialized in preparation of such arrays, such as Roche-Nimblegen.

After completion of the preparation of a set of probes, the probes may be fixed on the support to prepare a CGH array.

In one embodiment, a CGH array containing a set of probes for all the 62 genes in Table 1 was prepared.

This approach is consistent with the chosen technology, allowing the spot of up to 12×135 000 probes in the same array, thus identifying at a glance deletions or duplications in all the known genes with a high resolution (one probe every 10 bases or so).

Device B may be manufactured as follows:

Sequences of targeted genes known to be responsible for at least one of MD, LGMD, CMD and other neuromuscular diseases were obtained from the web site of the UCSC, and are shown in Table 1.

The genes on which analysis is implemented are selected from Table 1, as explained above.

A set of probes for Device B is designed by a well known technique of the state of the art, as explained above.

For example, sequence capture arrays which may be used in Process B may be manufactured by any manufacturer specialized in preparation of such arrays, such as Roche-Nimblegen or Agilent.

After completion of the preparation of a set of probes, the probes may be fixed on the support to prepare a sequence capture array.

In one preferred embodiment, a sequence capture array containing a set of probes for all the 62 genes in Table 1 was prepared.

This approach is consistent with the chosen technology, allowing the spot of 1×385 000 probes in the same array. The sequences captured by this array, are then desorbed and characterized by HTS. Advantage of HTS is that, for example, 10 patients could be pooled and sequenced simultaneously decreasing by 10 the cost of mutation identification.

The methods according to the invention have advantageous properties.

Process A allows determining a number of CNV(s) on at least 25 genes selected from the 31 genes of Group 1 in Table 1.

Process B allows determining a number of point mutation(s) on at least 25 genes selected from the 31 genes of Group 1 in Table 1.

A combined determination of Process A and Process B allows detecting all possible mutations on the targeted genes, and therefore determining a number of at least one of MD, LGMD, CMD and other neuromuscular diseases.

In particular, said combined determination allows increasing the rate of determination of a number of at least one of MD, LGMD, CMD and other neuromuscular diseases.

The present challenge lies in increasing determination rate via characterisation of all mutation types, allowing characterization of the genotype also in rare and atypical phenotypes, in genetically ambiguous sporadic cases and in NMDs whose pathophysiology is multiallelic or multigenic.

By a gene by gene approach analysis of the art of the technique, from 30% to 40% of patients suffering from NMDs are devoid of genetic confirmation. In contrast, by the analytic method of the present invention, the rate of non-analysed patients is usually less than 5%, when Process A and Process B are carried out on 62 genes.

Because of the special selection of the group of genes of Table 1, involved in at least one of MD, LGMD, CMD and other neuromuscular diseases, and of the specific Devices A and B both provided with a set of probes for said group of genes, the process of the present invention allows determining all possible mutations in said genes and therefore determining a number of at least one of MD, LGMD, CMD and other neuromuscular diseases, with a rate of determination of at least 90%, often at least 95%, and generally at least 99%, when Process A and Process B are carried out on all the 62 genes of Table 1.

A further subject matter of the present invention relates to a method for determining at least one of MD, LGMD, CMD and other neuromuscular diseases, comprising implementing the above mentioned step(s) of determination.

The method of the present invention can be applied to the determination of a number of CNV(s) or point mutation(s), or CNV(s) and point mutation(s) arising from at least one of MD, LGMD, CMD and other neuromuscular diseases.

In one embodiment of a method of identifying in vitro molecular causes of Inherited Neuromuscular Disorders of the present invention, step (ii) comprises only Process A.

In another embodiment of a method of identifying in vitro molecular causes of Inherited Neuromuscular Disorders of the present invention, step (ii) comprises only Process B.

In a preferred embodiment of a method of identifying in vitro molecular causes of Inherited Neuromuscular Disorders of the present invention, step (ii) comprises both Process A and Process B.

If one or more CNVs or one or more punctual mutations are(is) detected, the physiological sample comprising a genome of a subject is classified as positive and a precise type of at least one of MD, LGMD, CMD and other neuromuscular diseases is allotted to said sample.

The present invention provides sensitive and reliable tools for detecting at least one of MD, LGMD, CMD and other neuromuscular diseases with a high determination rate, as evidenced by the Examples.

Said determination rate is at least 90%, often at least 95%, and generally at least 99%.

With the genes of Table 3, the determination rate is at least 99%, and almost 100% in patients with NMDs whose prevalence is greater than or equal to 1/100000.

They allow determining all possible mutation(s) involved in at least one of MD, LGMD, CMD and other neuromuscular diseases, in targeted gene(s) in only a single step process (2,100,000 probes).

This is possible due to the specificity of the selection of the group of genes of Table 1, involved in at least one of MD, LGMD, CMD and other neuromuscular diseases, and the specificity of Device A and Device B, both provided with a set of probes for said groups of genes, with a high determination rate. Said determination rate is at least 90%, often at least 95%, and generally at least 99%, when Process A and Process B are carried out on all the 62 genes of Table 1.

The process of the present invention increases the ratio of precisely analysed patients (either new analysed patients or reoriented patients for which initial analysis was erroneous).

Therefore the process of the present invention allows improving genetic counseling and patient management, establishing phenotype-genotype correlations, constructing dedicated databases and including patients in current or future clinical trials due to the special selection of groups of genes to be analysed.

The present invention allows also reducing analysis costs by the special selection of group of genes to be analysed, and by using platforms with high analytic capacities. The method of the present invention really corresponds to a “one-shot” technology that considerably reduces both the time and the cost of the whole analytic process.

Such an approach also dramatically reduces the costs associated to analysis on the medium term. On the long term this contributes to decreasing the global prevalence of NMDs by developing a suitable analytic counseling.

The special selection of groups of genes to be analysed allows reducing the time-to-analysis (down to 72 h to one week) for patients and families.

One must keep in mind that patients with a well characterized pathology, both on clinical and genetic sides, are the only one eligible for clinical trials or protocols, which become more and more numerous.

The scope of the invention can be understood better by referring to the examples given below, the aim of which is to explain the advantages of the invention. The invention will now be described by means of the following Examples.

### DESCRIPTION OF THE DRAWINGS

FIG. 1 shows a flow chart depicting the major steps involved in detecting at least one of MD, LGMD, CMD and other neuromuscular diseases, using the gene by gene exploration of the prior art.

FIG. 2 shows a flow chart depicting the major steps involved in detecting at least one of MD, LGMD, CMD and other neuromuscular diseases using a method of the present invention.

FIG. 3 shows the result of analysis of a sample taken from one uncharacterized male LGMD patient, using a CGH array.

FIG. 4 shows the result of analysis of a sample taken from one uncharacterized female LGMD patient, using a CGH array.

FIGS. 5 and 6 show the result of analysis of DNA from 2 patients, using CGH array showing new candidates genes for LGMD.

FIG. 7 shows the results of identification of a genomic variation in a patient using sequence capture and Illumina sequencing.

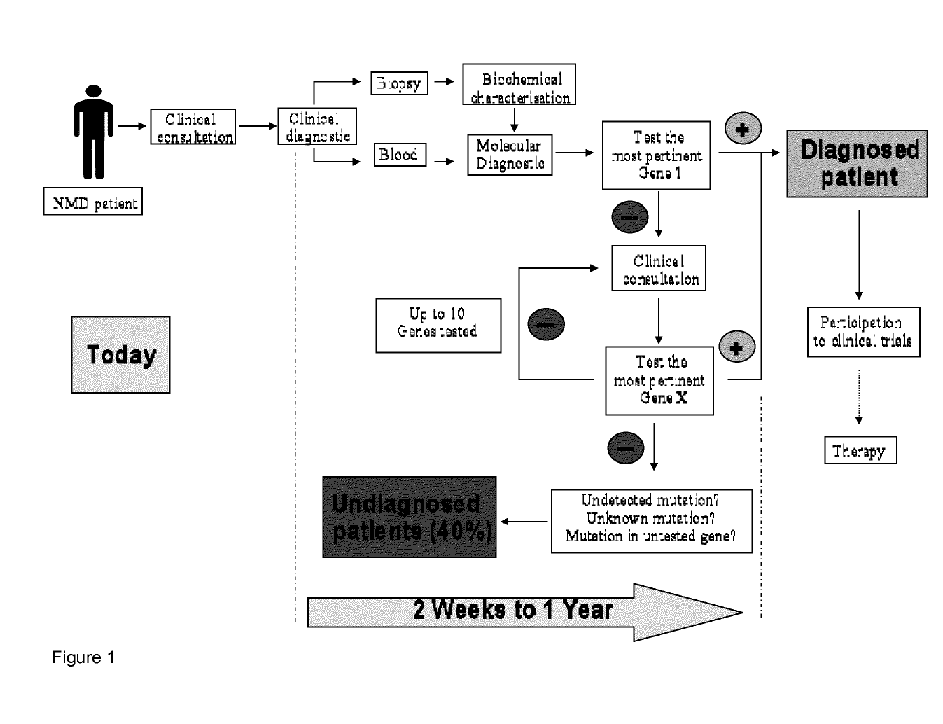

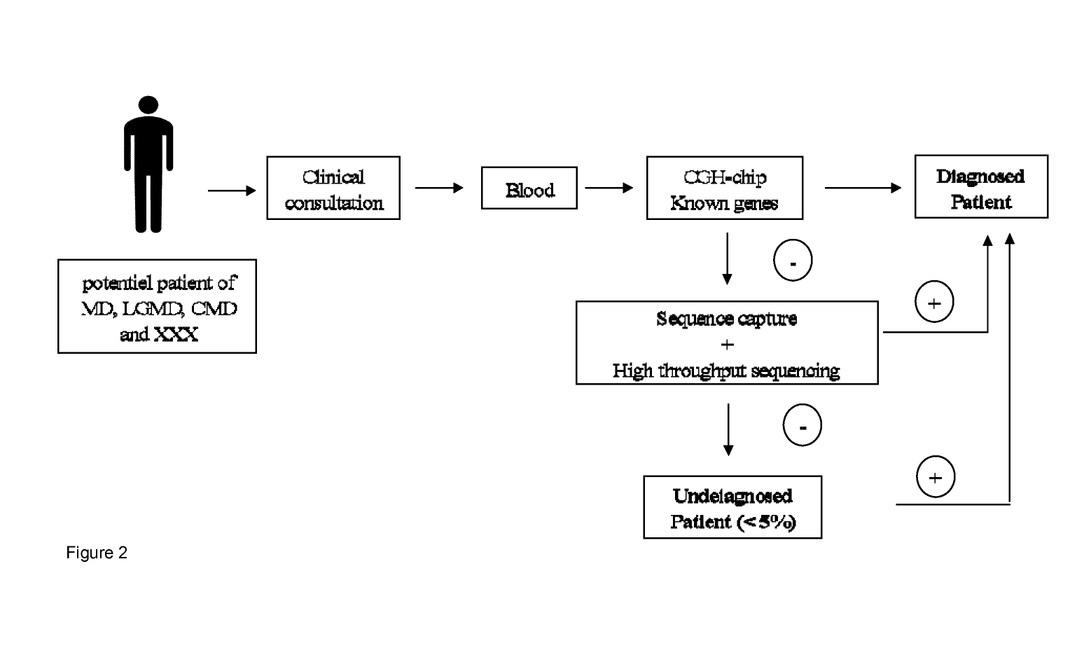

### EXAMPLES

### Example 1

### Manufacture of Devices

A CGH array and a sequence capture array ware prepared by Digital Mirror Device according to methods known in the art. The Digital Mirror Device creates “virtual masks” that replace physical chromium masks used in traditional arrays.

These “virtual masks” reflect the desired pattern of UV light with individually addressable aluminium mirrors controlled by the computer. The DMD controls the pattern of UV light projected on the microscope slide in the reaction chamber, which is coupled to the DNA synthesizer. The UV light selectively cleaves a UV-labile protecting group at the precise location where the next nucleotide will be coupled. The patterns are coordinated with the DNA synthesis chemistry in a parallel, combinatorial manner such that 385,000 to 4.2 million unique probe features are synthesized in a single array.

The set of probes for CGH array has been selected according to the following rules:

probes cover gene+/−2 kb

average probe density is 1/50

probes are alternated on (+) and (−) strands

with a tiling of
10 pb tiling in exonic regions and intron-exon boundaries (150 bp upstream and downstream of the exon)
30 pb tiling in 3′ and 5′ UTR
one probe for 100 bp in introns

backbone probes each 6 kb

total number of probes: 137207
gene probes (exon, intron, 5′ and 3′-UTR): 69570
backbone probes: 67637 probes (one each 6 kb on average).
The set of probes for sequence capture array has been selected according to the following rules:

exonique regions apart from 3′UTR are covered,

exonic regions and intron-exon boundaries (200 bp upstream and downstream of the exon) are covered,

1 kb upstream and downstream of each gene (5′ and 3′ UTR) are covered, and

probes are alternated on (+) and (−) strands.

### Example 2

### CGH Array NimbleGen

### Starting Material:

Genomic DNA from patients is used as starting material for CGH analysis. It will be compared to a reference DNA corresponding to a pool of anonymous donors (Promega, G1521).

The extraction protocol recommended for DNA purification is the Qiagen DNeasy Blood & Tissue Kit. (Qiagen, 50x, cat. no. 69504). Optional RNase treatment step must be achieved for these applications (RNase A, 100 mg/ml, cat. no. 19101).

### Quality Control:

DNA are assessed for quality and concentration using respectively agarose gel and spectrophotometer method (Nanodrop, ND-1000). RNase A treatment is recommended as RNA contamination could interfere during hybridization.

The quality control of genomic DNA is based on Nanodrop spectrophotometer measurements. Absorbance at 260 nm (A260) is used to assess quantity and A260/A280, A260/A230 ratios are calculated to assess purity of samples.

These ratios should be as follows: A260/A230≧1.9
A260/A280≧1.8

To determine integrity of DNA, 250 ng should be analyzed on a 1% agarose gel to ensure that they show no sign of RNA contamination or degradation.

### DNA Labelling:

Each genomic DNA from patients is labelled using known fragment allowing incorporating cyanin 3 (Cy3) in the DNA. In the same time, reference DNA is labelled using the same method except that cyanin 5 (Cy5) is incorporated instead of Cy3.

### Hybridization:

Same quantity of labeled DNA is then pooled (1 DNA from patient with the reference DNA) in a suitable hybridization cocktail and added into a hermetic chamber on the array. Hybridization is led during 3 days at 42° C. using the NimbleGen hybridization Station.

### Washing and Scanning:

Arrays are washed using several buffers by increasing stringency and finally dried. Slides are then scanned twice in a 2 μm-resolution scanner (MS200 NimbleGen) using 2 wave-lines corresponding to each fluorochrome.

### Data Analysis:

Quality control check and grid alignment are performed on scanned images. NimbleScan software is then used to convert intensity into raw data files and calculate log 2 (ratio) corresponding DNA from patients normalized by the reference. Ratio data are in .gff format file allowing visualizing the results using a genome browser (SignalMap) or other third part tools (like CGH-web).

For a detailed protocol see: http://www.nimblegen.com/products/lit/NG_CGHCNV_Guide_v8p0.pdf

### 2-1: Deletions in DMD (Process A)

A blood sample was taken from one male patient supposed to be suffering of LGMD. This sample was analysed with a CGH array provided with a set of probes for the 62 genes of Table 1.

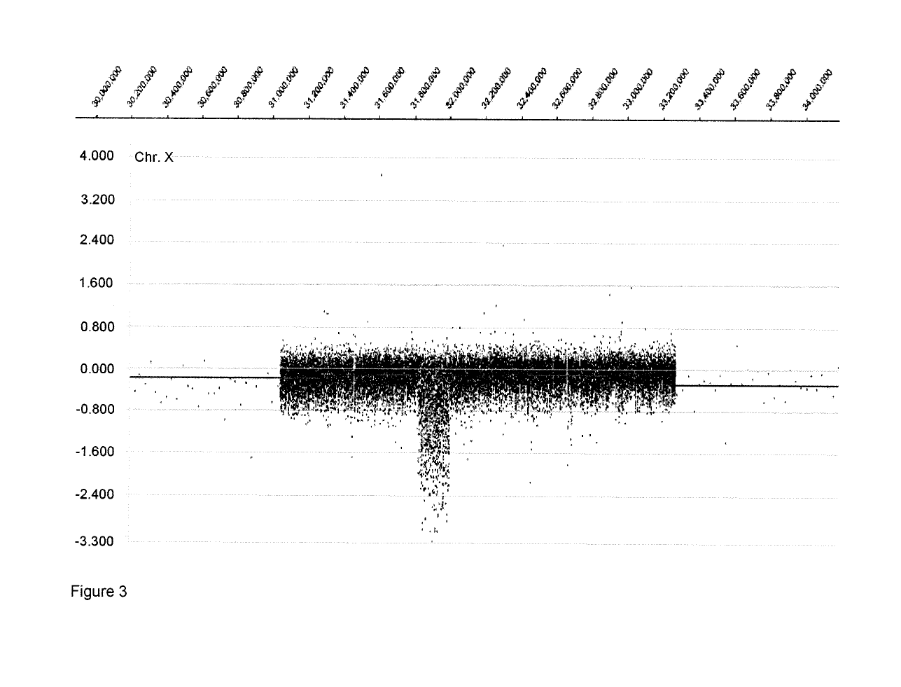

As shown in the signal map visualization of FIG. 3, a hemizygous deletion of exons 45 to 47, genomic size: 173.020 bp was detected in the sample.

In FIG. 3, the abscissa indicates genomic coordinates, and the ordinate indicates the log 2 ratio of signals.

The breakpoints determined by CGH are ChrX: 31827185-32000205. The deletion by qPCR and delimited the breakpoints in a 2 kb interval was confirmed (chrX: 31827185-2000-32000205+774).

The deletion results in an in-frame deletion: c.6439_{—}6912del (p.Glu2147-Lys2304del).

This result indicates that the male patient supposed to be suffering of LGMD is in fact attained by DMD.

### 2-2: Deletions in DMD (Process A)

A blood sample was taken from one female patient supposed to be suffering of LGMD. This sample was analysed with a CGH array provided with a set of probes for the 62 genes of Table 1.

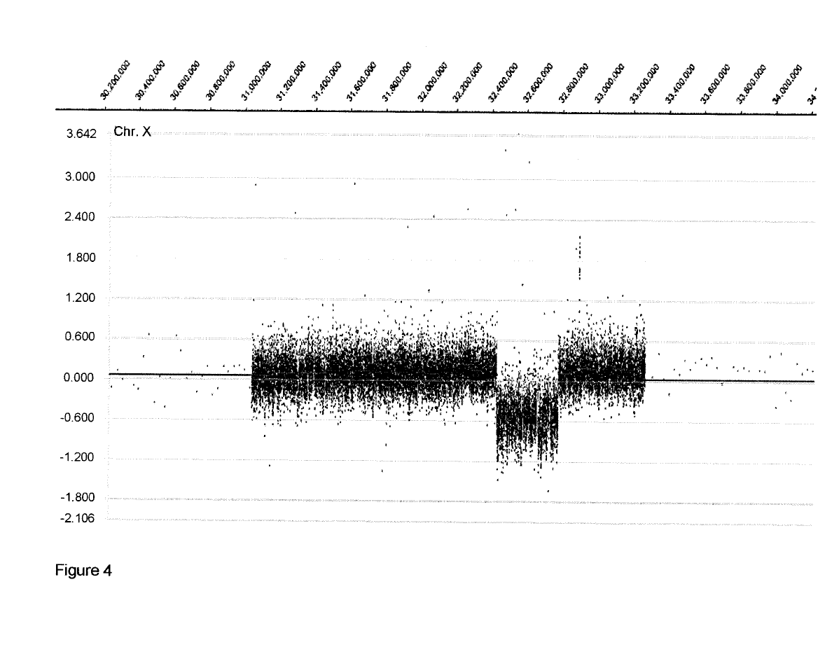

A heterozygous deletion of exons 3 to 18 genomic size: 346.847 bp was detected in the sample (FIG. 4).

In FIG. 4, the abscissa indicates genomic coordinates, and the ordinate indicates the log 2 ratio of signals.

The breakpoints determined by CGH are ChrX: 32432158-32779005. The deletion was confirmed by qPCR and delimited the breakpoints in a 1 kb interval (ChrX: 32432158(−1276)−32779005(−365). The deletion results in an in-frame deletion in the protein: c.94_{—}2292del.

This result indicates that the non-analysed patient is in fact attained by DMD.

2-3: Potential MD Genes: C6orf142 Process A

A blood sample was taken from 4 patients supposed to be suffering of LGMD. These samples were analysed with a CGH array provided with a set of probes for the 62 genes of Table 1 plus some additional candidate genes for LGMD.

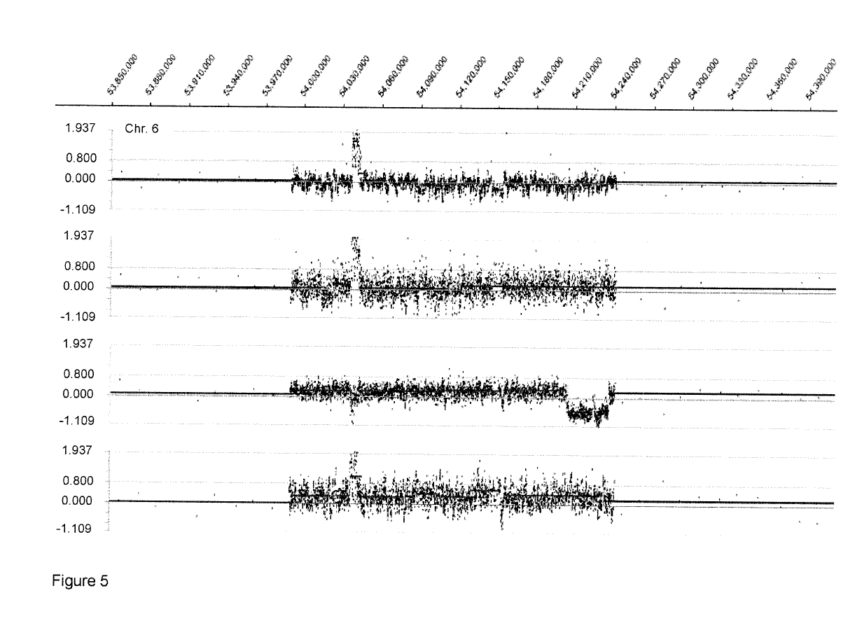

A homozygous deletion of around 30 kb (position around 54,210,000 to 54,540,000 of chromosome 6) was detected in one of the patients (FIG. 5, third line) containing 2 coding exons (exon 9 and 10) of C6orf142 gene. We can also notice the detection of a highly polymorphic CNV of less that 10 kb (position around 54,035,000 to 54,040,000) which illustrates the sensitivity of the method.

In FIG. 5, the abscissa indicates genomic coordinates, and the ordinate indicates the log 2 ratio of signals.

This 30 kb deletion is a potential mutation possibly responsible of LGMD in this patient. If this result is validated, C6orf142 would become a new gene involved in LGMD.

2-4: Potential MD genes: Cytoplasmic linker protein (CLIP)-170 (Process A)

A blood sample was taken from 2 patients supposed to be suffering of LGMD. These samples were analysed with a CGH array provided with a set of probes for the 62 genes of Table 1 plus additional candidate genes for LGMD.

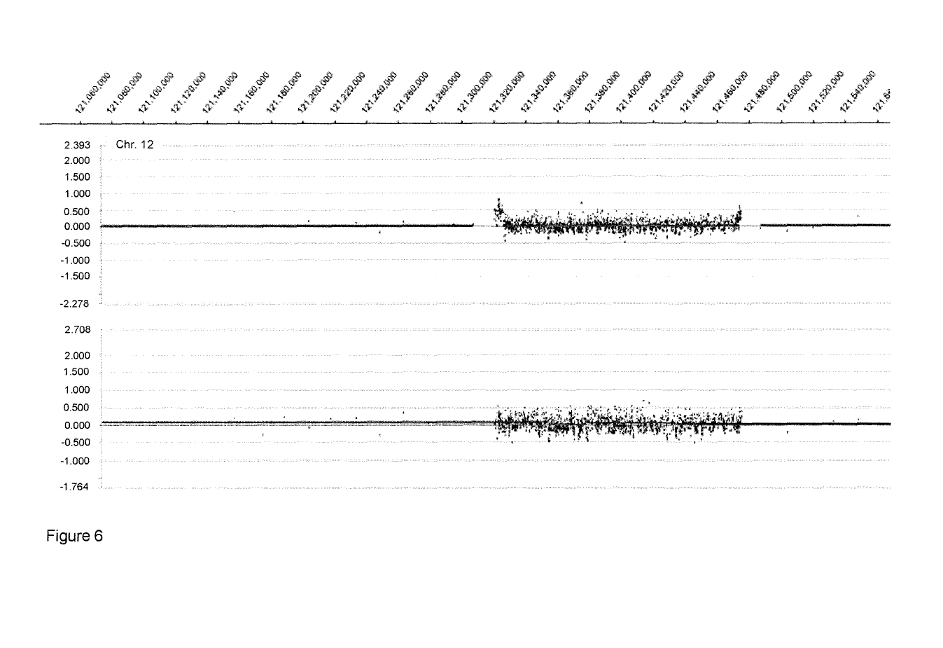

A 6-kb amplification was detected in the sample in line 1 (FIG. 6) in a region containing several coding exons (chromosome 12, around position 121,320,000 to 121,325,000).

In FIG. 6, the abscisa indicates genomic coordinates, and the ordinate indicates the log 2 ratio of signals.

Amplification of this region has been validated by qPCR indicated 2 additional copies in the patient genome. This variation in copy number in CLIP gene is a possible mutation that could be the genetic cause of LGMD in this patient.

### Example 3

### Sequence Capture Array (Process B)

In order to focus the sequencing on regions of interest, sequence capture strategies have been developed. In this aim, Agilent and Nimblegen propose in-solution capture methods that use specific oligonucleotide probes targeting regions of interest. These probes libraries are used to selectively capture DNA fragments from patients corresponding to these regions addressed by the sequencing. Here, next-generation sequencer HiSeq2000 from Illumina is used.

### Agilent Protocol:

(SureSelect Target Enrichment Kit, Agilent)

### Quality Control:

DNAs are assessed for quality and concentration using respectively agarose gel and spectrophotometer method (Nanodrop, ND-1000). RNase A treatment is recommended as RNA contamination could interfere during hybridization.

The quality control of genomic DNA is based on Nanodrop spectrophotometer measurements. Absorbance at 260 nm (A260) was used to assess quantity and A260/A280, A260/A230 ratios were calculated to assess purity of samples.

These ratios should be as follows: A260/A230≧1.9
A260/A280≧1.8

To determine integrity of DNA, 250 ng was analyzed on a 1% agarose gel to ensure that they show no sign of RNA contamination or degradation.

### DNA Preparation:

Genomic DNAs from patients were fragmented using Covaris device to obtain fragments between 150 and 200 bp length. Then, DNAs were repaired in order to get blunt-ended fragments that will be subjected to an addition of 5′-Phosphate before an 3′-end adenylation step. These modifications allow ligating specific adaptors (TruSeq adaptors, Illumina) that will be used during the sequencing step. To obtain enough material for the hybridization step, a PCR amplification using Herculase II Fusion DNA Polymerase (Ozyme) was performed.

The extraction protocol recommended for DNA purification is the Qiagen DNeasy Blood & Tissue Kit. (Qiagen, 50x, cat. no. 69504). Optional RNase treatment step must be achieved for these applications (RNase A, 100 mg/ml, cat. no. 19101).

Hybridization on in-Solution Library Probes:

DNA fragments were then hybridized 24 h at 65° C. in presence of the capture probes.

### Purification of Captured DNA:

Magnetic beads selection allows discarding non specific DNA and eluting the DNA fragments of interest. At the end of this step, a PCR amplification step was performed in order to amplify the material and to incorporate the specific TruSeq index sequences (Illumina). This barcode system will allow pooling DNA from different patients and sequencing all of them in a single sequencing run.

### Quantity Assessment:

Quantification was performed using qPCR kit (NGS Library Quantification, Agilent) in order to pool the samples in equimolar quantity. The pool was then ready for sequencing on HiSeq2000 platform.

For a detailed protocol see: http://www.chem.agilent.com/Library/usermanuals/Public/G7530-90000_SureSelect_IlluminaXTMultiplexed_v.1.2.pdf

### NimbleGen Protocol: (SeqEZ Library, NimbleGen)

Protocol is very similar to Agilent SureSelect except for the following few points:

Fragmentation must be between 200 and 400 bp length.

The TruSeq indexes are added before hybridization by ligation.

Amplification is done using Fusion HF PCR master mix (Ozyme).

Hybridization is performed at 47° C. for 72 h.

Enrichment is assessed using qPCR methods to validate capture efficiency.

For a detailed protocol see: http://www.nimblegen.com/products/lit/06588786001_SeqCapEZLibrarySR_Gui de_v3p0.pdf

### 3-1. Identification of a Genomic Variation (Process B)

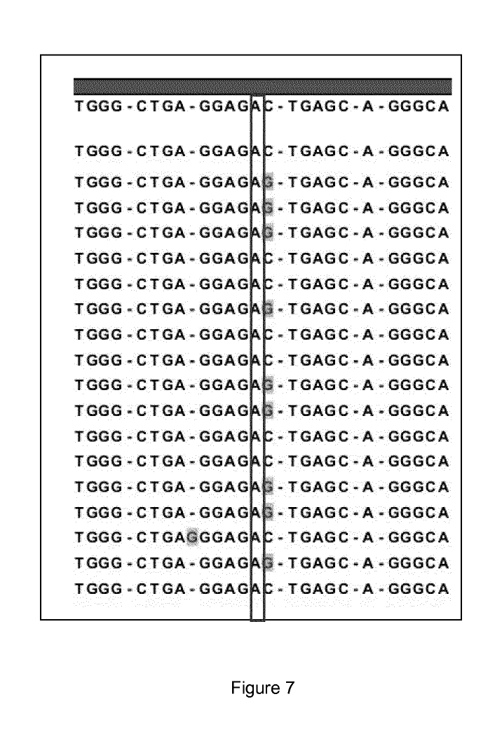

FIG. 7 shows the result of identification of a genomic variation in a patient using sequence capture and Illumina sequencing. Several reads from the sequencing of one patient (blue) have been aligned on the reference genome sequence (green). A variant heterozygous position has been identified (red) where a G allele is found in addition to the C corresponding to the reference sequence.

### 3-2. Sequencing (Process B)

Table 4 shows the results of a sequencing run led on Illumina HiSeq device using 2 DNA controls with known mutations (CTR-1 and CTR-2) and 8 LGMD patients with unknown mutations.

TABLE 4
Results of a sequencing run led on Illumina HiSeq
device using 2 DNA controls with known mutations (CTR-1
and CTR-2) and 8 LGMD patients with unknown mutation

| Sample | Mutation | typeannot | Gene | chrom | Ref |
|---|---|---|---|---|---|
| CTR-1 | SNP | Nonsense | DMD | chrX | G |
| CTR-2 | Indel | Deletion | SGCA | Chr17 | C |
| CTR-2 | SNP | Missense | SGCA | Chr17 | G |
| LGMD-1 | SNP | Missence | TTN | Chr2 | T |
| LGMD-1 | SNP | Missence + | TTN | Chr2 | C |
| | | splice exon | | | |
| LGMD-2 | SNP | Nonsense | TTN | Chr2 | A |
| LGMD-2 | SNP | Missense | TTN | Chr2 | G |
| LGMD-3 | SNP | Missense | CAPN3 | Chr15 | A |
| LGMD-4 | SNP | Missense | NEB | Chr2 | C |
| LGMD-4 | SNP | Missense | NEB | Chr2 | C |
| LGMD-5 | Indel | Deletion | DYSF | Chr2 | G |
| LGMD-6 | SNP | Missense | TTN | Chr2 | C |
| LGMD-6 | Indel | Deletion | TTN | Chr2 | TTTCCTCTTCAGGAGCAA |
| LGMD-7 | Indel | Insertion | ANO5 | Chr11 | — |
| LGMD-7 | SNP | Missense | ANO5 | Chr11 | A |
| LGMD-8 | SNP | Nonsense | ANO5 | Chr11 | C |
| LGMD-8 | Indel | Insertion | ANO5 | Chr11 | — |

Results of a sequencing run led on Illumina HiSeq
device using 2 DNA controls with known mutations (CTR-1
and CTR-2) and 8 LGMD patients with unknown mutation

| Sample | Variant | statut | AA changes | Polyphen |
|---|---|---|---|---|
| CTR-1 | A | Het | Q > STOP | — |
| CTR-2 | — | Het | Frameshift (1D) | — |
| CTR-2 | A | Het | V -> M | Probably damaging |
| LGMD-1 | A | Het | D -> V | Probably damaging |
| LGMD-1 | T | Het | V -> M | Probably damaging |
| LGMD-2 | T | Hom | Y -> STOP | — |
| LGMD-2 | A | Hom | R -> C | Probably damaging |
| LGMD-3 | G | Het | Y -> C | Probably damaging |
| LGMD-4 | T | Het | G -> D | Probably damaging |
| LGMD-4 | T | het | R -> Q | Probably damaging |
| LGMD-5 | — | Het | Frameshift (1D) | — |
| LGMD-6 | T | Het | R -> H | Probably damaging |
| LGMD-6 | — | Het | Deletion (18D) | — |
| LGMD-7 | A | Het | Frameshift (1I) | |
| LGMD-7 | G | Het | N -> S | - probably damaging |
| LGMD-8 | T | Het | R -> STOP | — |
| LGMD-8 | GGAC | het | Frameshift (4I) | — |

The 3 known mutations in both controls DNA have been easily detected (stop codon in DMD gene for CTR-1 and 1 deletion and 1 pathogenic amino-acid substitution in SGCA gene for CTR-2). Concerning the 8 patients with no mutation described so far, 3 of them show mutations in TTN, 2 in ANO5, 1 in DYSF, CAPN3 and NEB. All these genes belong to the core list of 62 genes of interest as described in tables 1 and 2. These results illustrate the efficiency of the tool and the strength of the process B to identify point mutations.

### REFERENCES

(1) Piluso G, Dionisi M, Del Vacchio Blanco F, Torella A, Aurino S, Savarese M, Giugliano T, Bertini E, Terracciano A, Vainzof M, Criscuolo C, Politano L, Casali C, Santorelli F M, and Nigro V. Motor chip: a comparative genomic hybridization microarray for copy-number mutations in 245 neuromuscular disorders. Clin. Chem. 2011: 57:11 1584-1596.
(2) Kaplan J-C. The 2012 version of the gene table of monogenic neuromuscular disorders. Neuromuscular Disord. 21(2011) 833-861.
(3) Saillour Y, Cossée M, Leturcq F, Vasson A, Beugnet C, Poirier K, Commere V, Sublemontier S, Viel M, Letourneur F, Barbot J C, Deburgrave N, Chelly J, and Bienvenu T. Detection of exonic copy-number changes using a highly efficient oligonucleotide-based comparative genomic hybridization-array method. Hum. Mutat. 2008 September; 29(9):1083-90.
(4) Hegde M R, Chin E L, Mulle J G, Okou D T, Warren S T, Zwick M E. Microarray-based mutation detection in the dystrophin gene. Hum. Mutat. 2008 September; 29(9):1091-9.
(5) Shinawi M, Cheung S W. The array CGH and its clinical applications. Drug Discov Today. 2008 September; 13(17-18):760-70. Epub 2008 Jul. 17. Review.
(6) Technical note: Roche NimbleGen Probe Design Fundamentals. 2008 June (http://www.nimblegen.com/index.html

1. A method of identifying in vitro molecular causes of Inherited Neuromuscular Disorders, comprising the following steps:
(i) providing a physiological sample comprising a genome of a subject, and
(ii) implementing on said sample at least one of Process A and Process B, wherein
Process A is determining a number of copy number variation(s), with respect to a sample of a normal subject, on at least 25 genes selected from the 31 genes of Group 1 in Table 1; and
Process B is determining a number of point mutation(s), with respect to a sample of a normal subject, on at least 25 genes selected from the 31 genes of Group 1 in Table 1.

2. The method according to claim 1, wherein said step (ii) comprises only Process A.

3. The method according to claim 1, wherein said step (ii) comprises only Process B.

4. The method according to claim 1, wherein said step (ii) comprises Process A and Process B.

5. The method according to claim 1, wherein Process A or Process B is, or Process A and Process B are carried out on all the 31 genes of Group 1 in Table 1.

6. The method according to claim 1, wherein Process A or Process B is, or Process A and Process B are carried out on all the 31 genes of Group 1, and on at least 10 genes of Group 2 in Table 1.

7. The method according to claim 1, wherein Process A or Process B is, or Process A and Process B are carried out on all the 31 genes of Group 1, on all the 15 genes of Group 2, and on at least 5 genes of Group 3 in Table 1.

8. The method according to claim 1, wherein Process A or Process B is, or Process A and Process B are carried out on all the 31 genes of Group 1, on all the 15 genes of Group 2, on all the 9 genes of Group 3, and on at least 4 genes of Group 4 in Table 1.

9. The method according to claim 1, wherein Process A or Process B is, or Process A and Process B are carried out on all the genes of the Table hereunder:

| | | | |
|---|---|---|---|
| ACTA1 | FKRP | BAG3 | DNAJB6 |
| ANO5 | FLNC | DAG1 | FKTN |
| BIN1 | GNE | LAMP2 | MYH2 |
| CAPN | LAMA2 | LDB3 | SGCB |
| CAV3 | LARGE | MTM1 | SGCD |
| COL6A1 | LMNA | NEB | CHKB |
| COL6A2 | MYOT | PABPN1 | CNTN1 |
| COL6A3 | POMT1 | PTRF | ITGA7 |
| DES | POMT2 | TNNT1 | PLEC1 |
| DMD | RYR1 | TPM2 | TCAP |
| DNM2 | SGCG | TPM3 | |
| DYSF | SGCA | VCP | |
| FHL1 | TTN | CFL2 | |

10. The method according to claim 1, wherein Process A is carried out with a Device A comprising a set of probes for said genes.

11. The method according to claim 10, wherein said set of probes for said Device A comprises:
probes evenly spaced by about 50 bp distance between two consecutive probes, which hybridize said gene plus a region of about 2000 bp at the 5′ and 3′ terminal exons, and
backbone probes not evenly spaced by about 1000 bp distance between two consecutive probes, which represent on average from 20 to 30% of the total probes on said device.

12. The method according to claim 10, wherein said set of probes are manufactured according to the following rules:
probes cover gene+/−2 kb up and downstream
average probe density is 1/50
probes are alternated on (+) and (−) strands
with a tiling of
10 pb tiling in exonic regions and intron-exon boundaries (150 bp upstream and downstream of the exon)
30 pb tiling in 3′ and 5′ UTR
one probe for 100 bp in introns
backbone probes each 6 kb
total number of probes: 137207
gene probes (exon, intron, 5′ and 3′-UTR): 69570
backbone probes: 67637 probes (one each 6 kb on average).

13. The method according to claim 10, wherein said Device A for Process A is a Comparative Genome Hybridization array.

14. The method according to claim 1, wherein Process B is carried out with a Device B comprising a set of probes for said genes.

15. The method according to claim 14, wherein said set of probes for said Device B comprises:
probes of 70 to 120 pb, which hybridize all the exons of said genes with at least 2× tiling frequency.

16. The method according to claim 14, wherein said set of probes for said Device B for Process B is prepared according to the following rules:
exonic regions apart from 3′UTR are covered,
exonic regions and intron-exon boundaries (200 bp upstream and downstream of the exon) are covered,
1 kb upstream and downstream of each gene (5′ and 3′ UTR) are covered, and
probes are alternated on (+) and (−) strands.

17. The method according to claim 1, characterized in that Process B is carried out by a technique selected from the group consisting of Sequence capture, on-chip capture and in-solution capture.

18. The method according to claim 17, characterized in that said Device B is a Sequence capture array.

19. The method according to claim 1, characterized in that High Throughput Sequencing is used in Process B.


SEQUENCE LISTING (4 sequences)

SEQ ID NO: 1 - DNA, 18 bp, Homo sapiens
tttcctcttcaggagcaa

SEQ ID NO: 2 - DNA, 25 bp, Homo sapiens
tgggctgaggagactgagcagggca

SEQ ID NO: 3 - DNA, 25 bp, Homo sapiens
tgggctgaggagagtgagcagggca

SEQ ID NO: 4 - DNA, 26 bp, Homo sapiens
tgggctgagggagactgagcagggca